(19) United States
(12) Patent Application Publication
(10) Pub. No.: US 2022/0018069 A1
(43) Pub. Date: Jan. 20, 2022

(54) FRACTIONATION OF CRUDE TALL OIL

(71) Applicant: Stora Enso OYJ, Helsinki (FI)

(72) Inventor: Jari Kavakka, Stockholm (SE)

(73) Assignee: Stora Enso OYJ, Helsinki (FI)

(21) Appl. No.: 17/297,566

(22) PCT Filed: Dec. 6, 2019
(86) PCT No.: PCT/IB2019/060503

§ 371 Date: May 27, 2021

(30) Foreign Application Priority Data
Dec. 11, 2018 (SE) 1851548-6

(51) Int. Cl. D21C 11/00; C11B 13/00; C07J 9/00; B01D 15/18; C09F 1/02

(52) U.S. Cl. CPC D21C 11/0042; C11B 13/005; B01D 2215/023; B01D 15/185; C09F 1/02; C07J 9/005

(57) ABSTRACT

The present invention relates to fractionation of crude tall oil, which originates from the Kraft process black liquor. In the method, according to the present invention, simulated moving bed (SMB) chromatography is used to efficiently separate fractions from the crude tall oil.

### FIELD OF THE INVENTION

The present invention is directed to fractionation of crude tall oil, which originates from the Kraft process black liquor. In the method, according to the present invention, simulated moving bed (SMB) chromatography is used to efficiently separate fractions from the crude tall oil.

### BACKGROUND

During production of Kraft pulp, black liquor is formed and removed from the produced pulp. The removed black liquor comprises soap which needs to be separated from the black liquor since the soap comprises valuable raw materials. Another reason to separate the soap from the black liquor is that the soap may cause problems during subsequent treatment steps of the black liquor.

The separated soap comprises extractives, water, lignin, inorganic compounds, fibers and some black liquor. The fatty and resin acids of CTO are in the form of sodium salts in the soap. The amount of each component in the soap depends on the raw material, as well as seasonal variations thereof, used pulping process and on the process in which the soap is separated from the black liquor, i.e. the soap skimming process. The CTO is mainly composed of fatty and resin acids and unsaponifiables.

Crude tall oil is a valuable raw material and it is important to recover as much of the crude tall oil from the soap as possible. Crude tall oil can be used as a raw material for various chemicals and other products, e.g. biodiesel or detergents.

It is possible to separate the CTO from the soap by addition of an acid to the soap at certain temperature. After mixing of the soap and the added acid, tall oil is formed and it then separates into three major phases due to density differences of the phases; a CTO phase, a lignin phase and a spent acid phase, also referred to as brine. The lignin and spent acid phase are rejects in the CTO production and they need to be separated well from the CTO phase during the recovery of the CTO.

The amount of acid needed to separate the optimal amount of CTO from the soap depends on the quality of the soap, e.g. the CTO content, the water content, the fiber amount, the lignin content and/or the black liquor content. Today it is common to measure the density of the soap, and the pH and density of the spent acid as a measure of the amount of acid and water that needs to be added to separate the optimal amount of the CTO from the soap. These measurements are done online and the needed amount of acid and water is thereafter adjusted, i.e. feedback control.

Traditionally, CTO is fractionated using vacuum distillation to fractions like heads (light, low boiling compounds), fatty acids, rosin acids, and pitch (distillation residue). Also, due to similar boiling points of fatty and rosin acids, a middle fraction can be collected to prevent contamination of fatty and rosin acid fractions. During the distillation of CTO at high temperature alcohols are esterified with carboxylic acids resulting in lower yield of the free acid fractions and increase in the lower value pitch fraction. Furthermore, thermal decomposition of compounds may occur during high temperature distillation.

As described above, the CTO can be used for production of several different products. Alternatively, the CTO could be first separated into unsaponifiables and high acid number CTO. The high acid number CTO can be further separated into rosin acids and fatty acids. The unsaponifiables fraction contains i.a. phytosterols.

Phytosterols have several uses, including the use as food additives and as precursors for steroids. Several methods have been reported for the isolation of sterols from tall oil soap, such as the extraction of neat soap with a variety of organic solvents.

Currently, phytosterols are commercially produced e.g. from tall oil pitch. Due to the ester formation during distillation, phytosterol esters must be hydrolyzed if production of free phytosterols is targeted. This requires additional process steps.

### SUMMARY OF THE INVENTION

It has surprisingly been found that the method according to the present invention can be used to more efficiently separate CTO into one neutral fraction and one neutral depleted fraction. The neutral fraction mainly contains components generally described as unsaponifiables. The neutral depleted fraction mainly contains components such as high quality CTO, also referred to as high acid number CTO.

Thus, the present invention is directed to a process for separating components from crude tall oil comprising the steps of
a) providing crude tall oil,
b) subjecting the crude tall oil to simulated moving bed chromatography, thereby separating crude tall oil into at least two fractions, and
c) recovering each of the fractions, wherein each fraction contains at least one component.

The present invention is also directed to fractions recovered according to the process of the present invention.

### DETAILED DESCRIPTION

The simulated moving bed (SMB) chromatography used in step b) is continuous, i.e. the process of separating crude tall oil into at least two fractions is done continuously. A sequential simulated moving bed chromatography (SSMB) system has two or more columns that may be identical, which are connected to a mobile phase pump, and each other, by a multi-port valve. The configuration is such that all the columns are connected serially, forming a single continuous loop. Preferably, between each column there is one valve, thereby enabling one of four process streams: incoming feed mixture, exiting purified fast eluting component (raffinate), exiting purified slow eluting component (extract), and incoming washing solvent or eluent. Each process stream (two inlets and two outlets) proceeds in the same direction after a set time.

The washing solvent used in the chromatography according to the present invention is an organic solvent capable of solvating CTO compounds. Preferably, the solvent used in the chromatography is toluene, ethyl acetate, acetone, butanone, ethanol, methanol, propanol, isopropanol, n-butanol, xylenes, dichloromethane, chloroform, propylene carbonate, ethylene carbonate, acetic acid or a mixture thereof.

The solid phase (stationary phase) used in the chromatography is preferably silica, alumina, zeolite or polymeric resin bearing polar groups. Preferably, the solid phase is polar, such as a zeolite.

Preferably, step b) initially provides separation into one neutral fraction and one neutral depleted fraction. The neutral fraction contains components generally described as unsaponifiables. The neutral depleted fraction contains components such as high quality CTO, also referred to as high acid number CTO.

The tall oil acid number can be determined using methods known in the art. One method of evaluating the quality of tall oil is to describe its acid number which is the amount of needed potassium hydroxide in milligrams to neutralize 1 g of CTO. As used herein, the term “high acid number CTO” means crude tall oil having an acid number of at least 170, such as at least 175 or at least 180.

From the neutral fraction, phytosterols are preferably separated from other neutral compounds, for example by distillation, extraction and/or crystallization, essentially using methods known in the art. If the neutral fraction contains some of the washing solvent, the solvent can be distilled off or alternatively be part of the precipitation/crystallization solvent system. Produced precipitate/crystals can be further purified by vacuum distillation or recrystallization or combination thereof, optionally followed by drying.

From the neutral depleted fraction, if the high-quality CTO contains washing solvent, that solvent can be distilled off and the residue recovered as high-quality CTO. Alternatively, HQ-CTO can be further fractionated to tall oil fatty acids and tall oil rosin acids with either a chromatographic system or by standard vacuum distillation. In one embodiment, the separation of fatty acids from rosin acids is carried out in an additional step of SSMB separation. In another embodiment, the high acid number CTO is first converted into a mixture of fatty acid methyl esters and rosin acids by esterification. The fatty acid methyl esters and rosin acids can subsequently be separated from each other using methods known in the art.

The term “phytosterol” is intended to mean a sterol derived from plants and encompasses all plant sterols and the saturated forms of phytosterols thereof (i.e., phytostanols). Plant sterols fall into one of three categories: 4-desmethylsterols (lacking methyl groups); 4-monomethylsterols (one methyl group); and 4,4-dimethylsterols (two methyl groups) and include, but are not limited to, sitosterol (e.g., [alpha] and [beta] sitosterol), campesterol, stigmasterol, taraxasterol, and brassicasterol. The term “phytostanol” is intended to mean a saturated phytosterol and encompasses, but is not limited to, sitostanol (e.g., [alpha] and [beta] sitostanol), campestanol, stigmastanol, clionastanol, and brassicastanol. Phytosterols isolated as described herein may be quantified by any means known in the art.

The phytosterol crystallization can be performed using methods known in the art, including cooling, concentration by removing some of the solvent by distillation, evaporation to dryness followed by introduction of a solvent or solvent mixture in which the phytosterols only dissolve at elevated temperature followed by cooling or through seeding with phytosterol crystals or by adding anti-solvent. Preferably, the washing solvent in the SSMB purification should be selected such that the CTO is fully dissolved in said solvent at a temperature suitable for use in the SMB, such as a temperature of 20-100° C., yet a solvent such that upon cooling of said solvent to approximately room temperature or below, phytosterols precipitate or crystallize from said solvent. The precipitation or crystallization may occur after a step of evaporating, such as distilling off, some of or all of said solvent. Alternatively, another solvent, such as an anti-solvent, may be added to facilitate precipitation or crystallization of the phytosterols, optionally in combination with seeding.

The process according to the present invention may be carried out as a batch process or as a continuous process.

Preferably, the crude tall oil is pre-processed before being subjected to simulated bed moving chromatography. The pre-processing preferably involves removal of fibers and any other components that may cause clogging of the chromatography system.

As an alternative to crude tall oil, gum rosin, also known as colophony or Greek pitch, can be used in the method according to the present invention.

In view of the above detailed description of the present invention, other modifications and variations will become apparent to those skilled in the art. However, it should be apparent that such other modifications and variations may be effected without departing from the spirit and scope of the invention.

1. A process for separating components from crude tall oil comprising the steps of
a) providing crude tall oil,
b) subjecting the crude tall oil to a simulated moving bed chromatography, thereby separating the crude tall oil into at least two fractions, and
c) recovering each of the at least two fractions, wherein each fraction contains at least one component.

2. The process according to claim 1, wherein one of the at least two fractions comprises mainly unsaponifiables.

3. The process according to claim 1, wherein one of the at least two fractions comprises mainly crude tall oil having an acid number of at least 170.

4. The process according to claim 1, wherein the simulated moving bed chromatography is sequential simulated moving bed chromatography.

5. The process according to claim 1, wherein a solvent used in the simulated moving bed chromatography is selected from a group consisting of: toluene, ethyl acetate, acetone, butanone, ethanol, methanol, propanol, isopropanol, n-butanol, xylenes, dichloromethane, chloroform, propylene carbonate, ethylene carbonate, acetic acid and mixtures thereof.

6. The process according to claim 1, wherein a solid phase used in the simulated moving bed chromatography is selected from a group consisting of: silica, alumina, zeolite, or polymeric resin bearing polar groups.

7. The process according to claim 1, wherein phytosterols are isolated from at least one fraction recovered.

8. The process according to claim 1, wherein a crude tall oil having an acid number of at least 170 is isolated from at least one fraction recovered.

9. A fraction separated and recovered according to the method of claim 1.

10. A process for separating components from crude tall oil comprising the steps of
a) providing crude tall oil,
b) subjecting the crude tall oil to a sequential simulated moving bed chromatography, thereby separating the crude tall oil into at least two fractions, and
c) recovering each of the at least two fractions, wherein each fraction contains at least one component,
wherein one of the at least two fractions comprises mainly crude tall oil having an acid number of at least 170.

11. The process according to claim 10, further comprising:
isolating the crude tall oil having an acid number of at least 170 from the one of the at least two fractions.
